(19) United States
(12) Patent Application Publication
(10) Pub. No.: US 2023/0329788 A1
(43) Pub. Date: Oct. 19, 2023

(54) Osteotomy Based Scan Body for Surgical Vector Capture on a Patient Specific Bone Structure

(71) Applicant: Joseph Amalfitano, Traverse City, MI (US)

(72) Inventor: Joseph Amalfitano, Traverse City, MI (US)

(21) Appl. No.: 17/719,367

(22) Filed: Apr. 13, 2022

(51) Int. Cl. A61B 34/10; G16H 20/40; A61C 1/08; A61C 7/00

(52) U.S. Cl. CPC A61B 34/10; G16H 20/40; A61C 1/084; A61C 7/002; A61B 2034/104

(57) ABSTRACT

An osteotomy based scan body apparatus for an accurately planned dental implant vector on a bone implant site in patient bone includes a main support member adapted to rest adjacent the patient bone. A bushing support structure extending from the main support member is resiliently connected to the main support member and extends away from the patient bone. A drill guide bushing includes a support structure slot and a drill guide portion, wherein the support structure slot is configured to engage the bushing support structure non-rotationally. The drill guide portion includes a loop member having a loop that encircles the implant vector for receiving a dental bur. At least one anchoring support is affixed to the main support member to anchor the main support member in place.

### BACKGROUND

### Field of the Invention

The present invention generally relates to dental implants and dental implant surgery. More specifically, the present invention relates to a completing a surgery procedure on patient specific bone anatomy for increased accuracy.

### Related Art

Over the past several years various surgical procedures have been adapted to a computer guided approach where patient data is presented to the surgeon in the form of a virtual environment. A virtual environment is created where surgical implements can be placed within a patient's specific anatomy virtually prior to surgery. One such procedure involves the placement of dental implants.

There are various ways surgeons can approach a particular case. Some surgeries such as complex implant placement may be completed “free hand.” In these cases, a surgeon makes ad hoc treatment decisions during the surgery. Alternatively, guided surgery may involve extensive planning prior to starting the surgery for a patient. Free hand surgery requires that a surgeon make quick decisions during the surgery. Decisions made at the spur of the moment may not be as thoughtfully planned out as if the surgeon had some time to think about possible complications and outcomes.

Pre-operative planning can help a surgeon identify individual challenges a patient's specific anatomy may present and can give surgeons time to think about the surgery in advance, including how to deal with the problems a given case may present before they arise. In some instances, a surgeon may elect not to begin or complete a surgery based on pre-op planning or may elect to refer the case to a surgeon with more experience. Using pre-operative planning, these decisions can help clinicians provide optimal treatment for each particular case.

Currently, guided dental implant surgery is planned in a completely virtual environment. Patient specific digital data is obtained and presented on a computer screen. A surgeon then places virtual implants on the screen. After the case is planned, the surgical guide is produced and used during the surgery. This method has various drawbacks, one of which is that the surgeon is forced to visualize certain aspects of the bone anatomy in a virtual fashion, which may lack the nuance of observing a physical specimen.

Hence, what is needed is a system that allows a surgeon to complete a surgical procedure on patient's specific bone anatomy prior to surgery for improved patient outcomes.

### SUMMARY

An osteotomy based scan body apparatus for an accurately planned dental implant vector on a bone implant site in patient bone is shown and described. The apparatus includes, among other features, a main support member which is configured to rest adjacent the patient's bone, which is customarily the maxillary bone or mandibular bone, and may also be proximal the patient's zygoma. A bushing support structure, or a plurality of bushing support structures, extend from the main support member, and are resiliently connected to the main support member, generally extending away from the patient's bone receiving dental implants.

The apparatus also includes a drill guide bushing, or a plurality of drill guide bushings, the number corresponding to the number of bushing support structures. The drill guide bushing (or bushings) each have a support structure slot and a drill guide portion. Each of the support structure slots is configured to securely yet removably engage each of the bushing support structures in a non-rotational manner. Each of the drill guide portions includes a loop member, typically extending away from the support structure slot, and having a loop at the end. The loop encircles the implant vector, and is configured for receiving a dental bur. Therefore, the drill guide bushings guide the dental bur along the implant vector to ensure the correct implant angle relative to the patient's bone.

In one alternative implementation, the apparatus also includes at least one anchoring support affixed to the main support member. The anchoring support is configured to engage the patient's bone, thereby anchoring the main support member in place relative to the patient's bone without moving. In some implementations, the one or more anchoring supports may include at least one drill hole for fixing the anchoring support to the patient's bone with screws or pins. In other contemplated implementations, the bushing support structure is affixed to the main support member using a support armature configured to space the bushing support structure away from the main support member.

In another implementation each of the bushing support structures includes a male support structure configured for engaging the support structure slot of the drill guide bushings. Each of the bushing support structures also includes a support base. Preferably each of the bushing support structures is configured such that the support structure slot of each of the drill guide bushings cannot slide past the support base. In another implementation, each of the drill guide bushings also includes a thread retainer portion. The thread retainer portion may be located opposite the drill guide portion of the drill guide bushing. Alternatively, the thread retainer portion may be located laterally relative to the drill guide portion of the drill guide bushing. In another implementation, the apparatus includes a maxillary sinus template having template windows for locating maxillary sinus windows on the patient's maxilla. Preferably, the main support member is configured to abut the outside arch of the patient's maxilla.

In another implementation, the apparatus includes at least one palatal pad configured to engage the patient's maxillary bone opposite the main support member, thereby anchoring the main support member against the patient's maxilla. The one or more palatal pads may also include one or more palatal pad supports resiliently connecting each palatal pad to the main support member. In another implementation, the main support member is configured to engage or abut the outside arch of the patient's mandibular bone. In the mandibular implementation, at least one lingual pad is provided for engaging the patient's mandibular bone opposite the main support member, thereby anchoring the main support member in place. Each of the lingual pads may be mounted on a lingual pad support connecting the one or more lingual pads to the main support member.

In another implementation, the main support member may be a tooth supported framework which is configured to seat over a patient's existing teeth. The tooth supported framework preferably includes at least one cutout at the location of the dental implant vector to provide space for the drill guide bushings. In yet another implementation, the main support member is configured as a bite prosthetic jig. The bite prosthetic jig is configured to engage a patient's maxillary bone after the removal of all upper teeth and is also configured to include and hold in place a bite prosthetic.

### BRIEF DESCRIPTION OF THE FIGURES

FIG. 1 illustrates a perspective view of a segmented bone scan for a dental patient including a maxilla and zygoma.

FIG. 2 illustrates a perspective view of a physical 3D model derived from the segmented bone scan, including surgeon drilled osteotomies and scan bodies.

FIG. 3 illustrates a perspective bottom view of the 3D model including surgeon drilled osteotomies and scan bodies.

FIG. 4 illustrates a perspective bottom view of a right-side scan body model for a right side zygoma and pterygoid implant.

FIG. 5 illustrates a perspective front view of the right-side scan body model for a right side zygoma and pterygoid implant.

FIG. 6 illustrates a perspective front view of a stereolithographic printed scan model of a right side zygoma and pterygoid implant.

FIG. 7 illustrates a perspective front view of a right side zygoma implant model, having a right side zygoma and pterygoid framework installed thereon.

FIG. 8 illustrates a perspective front view of a right side zygoma implant model having a right side zygoma and pterygoid framework with drill guide bushings.

FIG. 9 illustrates a perspective view of a zygoma and pterygoid framework, including drill guide bushings.

FIG. 10 illustrates a maxillary sinus template for creating maxillary sinus windows during a zygomatic implant operation.

FIG. 11 illustrates the maxillary sinus template in position on the maxilla of a bone scan of a patient.

FIG. 12 illustrates a front view of a maxillary scan with scan bodies in place for a maxillary dental implant.

FIG. 13 illustrates a bottom view of the maxillary scan with scan bodies in place for the maxillary dental implant.

FIG. 14 illustrates a front view of the maxillary scan with a maxillary framework including bushing support structures.

FIG. 15 illustrates the top view of the maxillary framework including bushing support structures.

FIG. 16 illustrates the top view of the maxillary framework including bushing support structures and drill guide bushings.

FIG. 17 illustrates a top view of the maxillary bone with the maxillary framework including bushing support structures.

FIG. 18 illustrates a top view of the maxillary bone with the maxillary framework including bushing support structures and drill guide bushings.

FIG. 19 illustrates a bottom view of the maxillary framework including bushing support structures and drill guide bushings.

FIG. 20 illustrates a side view of the maxillary bone with the maxillary framework attached, including bushing support structures and drill guide bushings.

FIG. 21 illustrates a front view of a mandibular bone model including scan bodies.

FIG. 22 illustrates a bottom perspective view of a mandibular framework including bushing support structures.

FIG. 23 illustrates a top perspective view of a mandibular framework including bushing support structures.

FIG. 24 illustrates a perspective view of the mandibular bone model including the mandibular framework.

FIG. 25 illustrates a top view of the mandibular bone model including the mandibular framework and drill guide bushings in place.

FIG. 26 illustrates a perspective view of a tooth supported framework scan with scan body models and bushing support structures in place.

FIG. 27 illustrates a palatal view of a maxilla model, including scan bodies.

FIG. 28 illustrates a perspective view of a tooth supported framework, including bushing support structures.

FIG. 29 illustrates a perspective view of the tooth supported framework scan, with the tooth supported framework and drill guide bushings in place.

FIG. 30 illustrates a bottom view of a bite prosthetic jig with bushing support structures.

FIG. 31 illustrates a top view of a bite prosthetic jig with bushing support structures.

FIG. 32 illustrates a side view of a bite prosthetic jig and a bite prosthetic attached thereto.

FIG. 33 illustrates a perspective view of a patient bone scan with an attached bite prosthetic jig.

FIG. 34 illustrates a perspective view of a patient bone scan with an attached bite prosthetic jig and bite prosthetic.

FIG. 35 illustrates a perspective view of a scan body aligned with a bushing support structure.

FIG. 36 illustrates a perspective of the scan body aligned with a bushing support structure and a drill guide bushing.

### DESCRIPTION

The following description is presented to enable any person skilled in the art to make and use the invention and is provided in the context of a particular application and its requirements. Various modifications to the disclosed embodiments will be readily apparent to those skilled in the art, and the general principles defined herein may be applied to other embodiments and applications without departing from the spirit and scope of the present invention. Thus, the present invention is not limited to the embodiments shown but is to be accorded the widest scope consistent with the principles and features disclosed herein.

Working on a physical three-dimensional (3D) model allows a dental surgeon to prototype a surgery with an accurate patient-specific model. Prototyping the surgery provides a better feel for the surgery ahead of time, including giving the surgeon a sense of scale which cannot readily be appreciated using digital monitor techniques. Various views of a patient's anatomy can also be perceptually visualized, as not all views are capable of being seen using current computer algorithms. Using a patient-specific model, surgeons can hold a patient's specific anatomy in their hands without needing to visualize various aspects using their imagination.

Scan bodies are currently used in dentistry but mainly for implant orientation after an implant is placed. For example, after a dental implant is placed, a scan body is attached to the implant in the oral cavity and an impression or 3D scan is taken. The scan body is aligned with a pre-design matching design that provides an accurate 3D localization of the implant in relation to the oral cavity and surrounding teeth if present. The innovation disclosed herein uses the scan body concept but uses it to localize an implant vector prior to surgery by placing the scan body in an osteotomy that is drilled in a patient specific bone model.

In an initial action, cone beam computed tomography (CBCT) or computerized tomography (CT) (or similar) scan data for a patient is obtained using an adequate field of view. The scan data is then segmented, removing various scanned structures, resulting in the patient's specific bone data. This is a labor-intensive step that often requires a slice-by-slice identification of bone structures. Most current surgical guide software uses algorithms to segment bone structures which is often inadequate and/or error prone. Inadequate, algorithmically derived bone segmentation may leave gaps in the data, forcing a surgeon to guess at what is bone and what is soft tissue. This phenomenon is caused by soft tissue and soft or thin bone structures having similar densities, requiring detailed manual segmentation to determine where actual bone exists. Once bone structure data is fully extracted, a 3D model (a “initial bone model”) can be produced and exported for printing, for instance in a stereolithography (STL) format.

The resulting model/models may be used by a surgeon to perform a mock, or prototype surgery using a surgical kit similar to that used in an actual surgery. The kit may include various diameter drills up to a specific diameter necessary to accept the newly designed scan bodies described herein. For example, if a scan body osteotomy projection is designed to be 2.7 mm the final osteotomy drill may be drilled at 2.85 mm, although any combination can be used based on the objectives of the surgery and amount of passivity and/or accuracy needed.

The benefits of this planning workflow are that multiple implant vectors can be tried and adjusted as necessary on the printed model to avoid anatomic structures and provide optimal implant length and angulation. If any particular osteotomy is drilled in the model and the surgeon finds it erroneous or sub-optimal, additional material can be injected into the previously drilled osteotomy, light cured or self-cured, and another vector can be chosen and drilled once the additional material cures. This may be repeated for as many sites as needed for any individual case. Thus, multiple models may be easily generated, and multiple trial runs of the surgery may be completed prior to actual surgery. This greatly enhances surgical outcomes compared to free-hand surgery that can only be “planned” in any sense while the surgical field is open. Additionally, surgeries planned using this highly accurate patient-specific data approach with a physical model are also far more accurate and error free than using a computer only approach.

Once the osteotomies are completed on the prototype models, scan bodies are inserted into the model osteotomies and the model bearing the scan bodies (the “scan body model”) is scanned in a lab quality scanner or similar device using photogrammetry for example, to build a highly accurate 3D model. An STL mesh is generated and used for further processing. The original scan data (i.e., the unprepared bone) of the initial bone model is imported into the design software and the scan data of the scan body model is aligned to the initial bone model scan data using 3D surface matching techniques. Computer Aided Design (CAD) renderings in STL format are imported into the design software and aligned to the scanned in data. This procedure provides a digital workflow to build a support structure that will hold one or more bushings that will be used to hold a drill in the pre-planned vectors from the initial bone model “practice” osteotomies and will be used during the surgery.

Once the support structure is digitally fabricated, it is prepared and exported for 3D printing in a resilient material, such as resin or metal for example. The device is delivered to the surgeon, who can test fit it on the initial bone model and verify that the implant vectors coincide with the prototype surgery planned osteotomies. With sophisticated milling equipment, the support structure may be able to be milled out of a solid block of metal material such as titanium or cast using a standard dental casting technique using the printed initial bone model as a mold. The surgical guide can then be sterilized and used during the surgery.

The proposed support structure guide design provides an open field of view for a surgeon, which allows the surgeon to observe the drill as it penetrates the patient's bone. This design also allows for adequate irrigation since the support structure design does not obscure the osteotomy site. Many current surgical guides obscure the point of entry of the bur into a bone structure. In contrast, the implementation disclosed herein is cantilevered to allow better visualization. This guide, including any guide designed in the manner disclosed, can be formed to provide adequate visualization of the drill tip entering the proposed osteotomy for an implant or surgical screw.

The same process could be used in any orthopedic procedure where the surgeon wants to translate a prototype model surgery into a viable surgical guide for a procedure such as cervical spine surgery or other types of bone based surgery including knee replacement, hip replacement or other craniofacial surgery. It is anticipated scan bodies may be prepared in different preferred sizes based on the size of the drill used in prototype surgery. Additionally, support structures may be designed using an initial bone model that is segmented in variety of ways to support various drills during many osseous based surgeries such as cervical spine surgery for example, or other bone-based surgeries. It is anticipated that many different designs of scan bodies, support structures and drill guide sleeves could be envisioned.

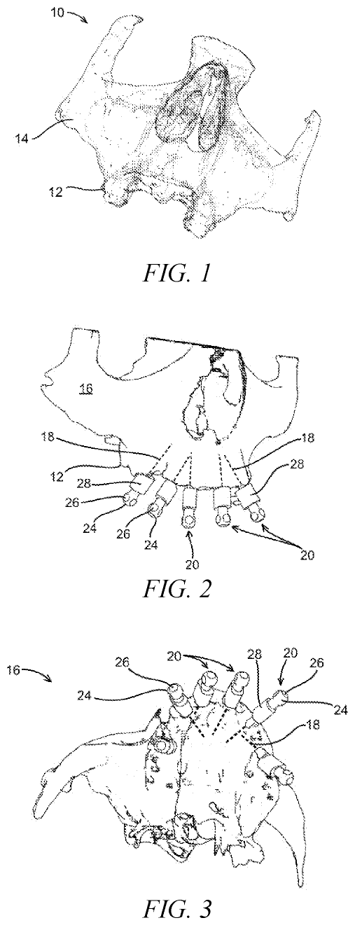

Referring to FIGS. 1-11, a framework design for placing zygoma implants is shown. Using this design, any zygoma configuration can be accomplished, from completely intra-sinus placement to extra-sinus placement. The design also allows other conventional implants to be placed and this example shows the additional placement of a pterygoid implant using burs or osteotomes. The framework design can be designed by drilling a 3D printed model and transfer of digital data using scan bodies as described elsewhere in this document or it may be completely designed within software.

FIG. 1 shows an exemplary initial scan 10, which is a pre-operative view of a patient's maxillary bone 12 and surrounding bone structures such as the zygoma 14 prior to preparation of the initial bone model (FIG. 2). The initial scan 10 is prepared as discussed above by obtaining cone beam computed tomography (CBCT) or computerized tomography (CT) (or similar) scan data for a patient. The initial scan 10 is then segmented by removing various scanned structures (not shown) such as soft tissue and non-osseous harder structures that may show up on the initial scan 10. Optionally, as discussed, by a slice-by-slice identification of bone structures such as the maxilla 12 and zygoma 14 is performed, resulting in the patient's specific bone data.

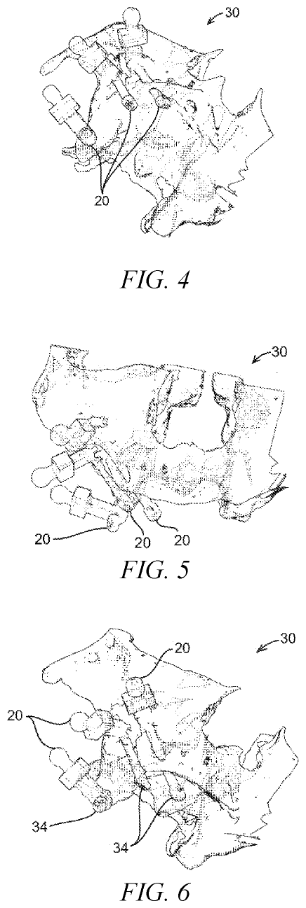

Referring to FIGS. 2 and 3, a bone model 16 is shown, having been exported from initial scan 10 data, as shown in FIG. 1. In the illustrated implementation, a bone model of the patient's maxilla 12 and zygoma 14 is shown. The bone model 16 is drilled by the surgeon with several osteotomies 18, and thereafter, scan bodies 20 are placed in the osteotomies 18. The scan bodies may need to be stabilized with glues or waxes to better stabilize it if needed on the printed model. The bone model 16 is then scanned with the scan bodies 20 in place (reflecting the position and angle of the osteotomies 18), to create a scan body model 30 (FIG. 4). The scan bodies 20 are inserted into the planned osteotomies 18 so that vectors (along the axis of each osteotomy) can be captured in a digital fashion. The scan bodies 20 are configured with a diameter that allows them to snugly fit into each osteotomy 18, thereby optimizing the vector captures of each osteotomy 18 previously drilled by the surgeon.

Still referring to FIGS. 2 and 3, the sample bone model 16 maxillary bone 12 structure is shown with six maxilla osteotomies 18 drilled in the bone model 16, and with six (6) scan bodies 20 inserted into osteotomies 18 that have been used to digitally capture the planned implant vectors. The implant vectors were chosen by observing and manipulating the bone model 16 and drilling into it to avoid undercuts and avoid vital structures such as naso-palatine structures or maxillary sinuses. These features can be readily visualized due to the hi-fidelity segmentation data used to create the bone model 16, and the transparent nature of the bone model 16 material. The material used for the bone model 16 allows for controlled drilling in similar nature to the real surgery.

The scan bodies 20 have spherical tops 24 including three flat sides 26 and a side left rounded, thereby pointed to where the drill guide bushings 50 (FIGS. 8 and 9) are going to be located. In one implementation, the scan bodies 20 may be made of polyether ether ketone (PEEK) or a similar thermoplastic material and milled on a five-axis mill for accuracy. The spherical tops 24, including the three flat sides 26, also have a rectangular filleted base 28 to allow for a 3D structure that can be used to align other components in CAD software. While this is one configuration the scan body design can be modified to increase accuracy or for different requirements of various bone based surgery.

Referring to FIG. 4, a bottom perspective right side scan body model 30 is shown having three scan bodies 20 in place, representing one half side (the patient's right side) of a planned zygoma implant surgery on the scan body model 30. The scan body model 30 (in this instance a maxillary bone) has been prepared by a surgeon simulating an optimal zygomatic implant placement. The implants can be placed in an extra-maxillary position or completely intra sinus, or any variation in-between. The scan bodies 20 are positioned on drill vector positioning guides 32, and the 3D scan position of the scan bodies 20 is used to transfer the implant vectors to software later used for framework design. The scan body model 30 shown in FIG. 4 illustrates a set up for two zygomatic implants and a dental implant in the pterygoid region.

Referring to FIG. 5, an alternative front perspective view of the right-side scan body model 30 is shown with three scan bodies 20 in place for capturing the insertion vectors of the two zygomatic implants and the single pterygoid implant. The scan bodies 20 are designed to attach to implant preparation burs thus capturing the insertion vector of the implants. The prototype maxilla scan body model 30 with the scan bodies 20 in place is then 3D printed and placed in a dental scanner wherein the 3D position of the scan bodies in relation to the bone structure is captured and used for downstream processing.

Referring to FIG. 6, a 3D stereolithographic representation of an actual patient is printed and given to the surgeon. The surgeon uses zygomatic implant surgical kit to prepare and place zygomatic implant vectors directly into the model. This way the surgeon can ensure that vital structures including the orbit and intra temporal fossa can be avoided. The pterygoid implant can also be modeled. Once this is completed drill lugs 34 are inserted into the prepared implant vectors and a scan body 20 is attached to each of the drill lugs 34. The entire model with the drill lugs 34 and scan bodies 20 can then be scanned using photogrammetry. 3D representation of the scan bodies 20 are then aligned to the scanned in scan bodies 20 and this allows support structures to be placed in the same vector as was prepared on the prototype model.

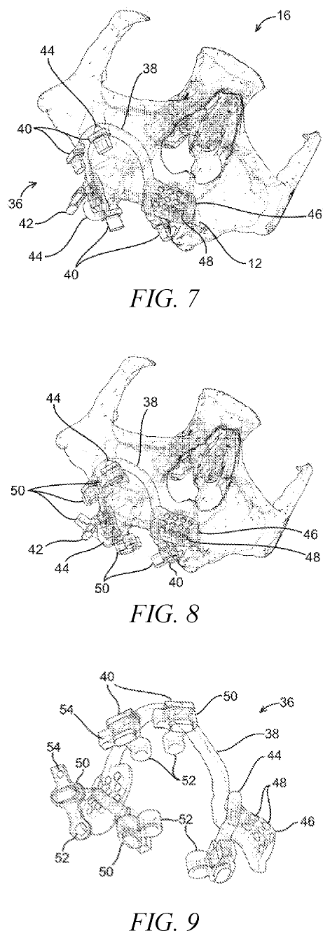

Referring to FIG. 7, a framework 36 is shown attached to the bone model 16 after placement of the insertion vectors of the implants using scan bodies 20 as shown in FIGS. 2-6. The example framework 36 shown in FIG. 7 is configured for placing zygoma implants. Using the framework 36, any zygomatic implant configuration can be accomplished, from completely intra-sinus placement extra sinus placement, relative to the maxillary sinus. The design also allows other conventional implants to be placed. Thus, the illustrated example framework 36 also shows the additional placement of a pterygoid implant using burs or osteotomes. The framework 36 design can be designed by drilling a 3D printed model and transferring digital data using scan bodies 20 (not shown) as described above. Additionally, it may be completely designed within software.

The framework 36 includes a main support 38. A series of bushing support structures 40 are provided for supporting drill guide bushings 50 (FIGS. 8 and 9). The bushing support structures 40, for zygoma implants, pterygoid implants, or other guide support structures in other configurations, are mounted on support armatures 44 which connect the bushing support structures 40 to the main support 38. The main support 38, and by extension support armatures 44, and the bushing support structures 40 extending therefrom, also includes one or more anchoring supports 46. The anchoring support 46 includes drill holes 48 for stabilizing the zygoma framework 36 on the patient. In the illustrated implementation, the anchoring support 46 is configured to engage the patient's maxillary bone 12.

Referring to FIG. 8, the framework 36 is shown with drill guide bushings 50 in place. Typically, two drill guide bushings 50 are needed to adequately guide the long zygoma implant burs into the patient's zygoma. By using two drill guide bushings 50, wobble is eliminated and adequate vector placement of the zygoma implants is ensured. The drill guide bushings 50 can be configured in a variety of sizes to accept any implant drill diameter. The drill guide bushings 50 can also be adapted to support tunneling or channeling burs for extra sinus placement of zygoma dental implants.

Referring to FIG. 9, the framework 36 is shown isolated from the initial bone model 16 (FIGS. 7 and 8). In the illustrated implementation, a top view of the framework 36 is shown with the zygoma guide support structures 40, pterygoid guide support structures 42 and drill guide bushings 50 in place. There are two drill guide bushings 50 for each zygomatic implant vector and one drill guide bushing 50 for the pterygoid implant placement. As discussed, two drill guide bushings 50 are necessary for the zygomatic implant due to the long drills used during the procedure. The design of the framework 36 allows maximum visualization of the surgical site and minimizes the need for reflection of soft tissue. Two anchoring supports 46 are provided that intimately rest on bone structure and the various drill holes 48 can be used to place bone fixation screws or pins (not shown). Several drill holes 48 are provided on each anchoring support 46 since secondary fixation screws (not shown) may need to be placed, due to the soft nature of bone.

Still referring to FIG. 9, in the illustrated implementation, each of the drill guide bushings 50 are configured as a sleeve that securely fits on each of the zygoma guide support structures 40 (and the pterygoid guide support structure 42) in a manner that prevents any rotational movement between the two. A drill guide portion 52 is configured to align a dental bur along the implant vector. In instances where two drill guide bushings 50 are used for a single implant, as is the case for those mounted on the zygoma guide support structures 40, the drill guide portions 52 align along the implant vector. Different drill guide bushings 50 can be used by removing them from the guide support structures 40, 42, based on the implant drill sequence used, and the drill guide bushings 50 can be milled such that the guide portions have any preferred diameter. Additionally, the distance from the guide portion 52 to the guide support structure 40, 42 can be of any length, and varied based on the depth of the zygoma implant vector within the maxillary sinus. This design allows any zygomatic implant to be placed including those that are entirely extra-sinus and those completely within the sinus cavity. A thread retainer portion 54 is configured to retain a thread-like tether (not shown) to retrieve a drill guide bushing 50 if it dislodges from a guide support structure 40, 42.

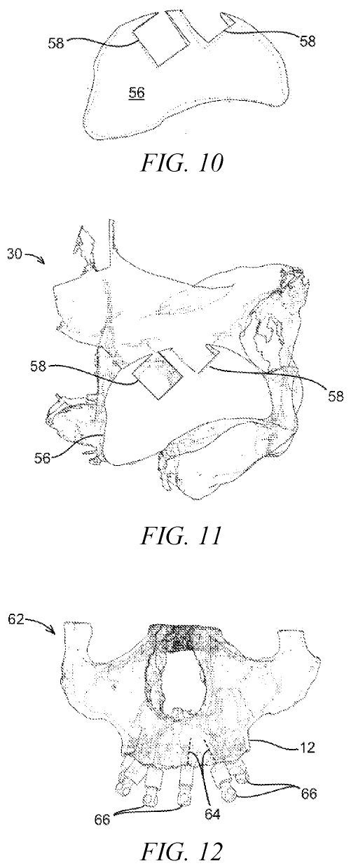

Referring to FIGS. 10 and 11, a maxillary sinus template 56 is used to create two maxillary sinus windows 58, prior to fixation of the framework 36. Although two maxillary sinus windows 58 are shown in the illustrated embodiment, additional windows may be created as needed. When the framework 36 is affixed on a patient, the drill guide bushings 50 located closer to a patient's zygoma need to extend into the patient's maxillary sinus. The drill guide bushings 50 are designed in various lengths depending on how deep the zygomatic implant is placed within the sinus and various diameters based on the drill diameters used throughout a procedure. In order to extend into the patient's maxillary sinus, maxillary sinus windows 58 are needed. In order to locate the maxillary sinus windows 58 at the proper location, the maxillary sinus template 56 is used. The maxillary sinus template 56 is formed on the bone model 30, either physically, or in design software.

Once the location of the drill guide bushings 50 (FIG. 9) needing access to the maxillary sinus are known, using the scan body model 30 or model data, the location of the maxillary sinus windows 58 can be ascertained. Once the location of the maxillary sinus windows 58 is ascertained, the maxillary sinus template 56 can be created with maxillary sinus windows 58 at the location of the drill guide bushings 50. The maxillary sinus template 56 may be created free form around the maxillary template windows 58 and is preferably formed in a shape allowing registration of the maxillary sinus template 56 in a specific location, such that misalignment is readily detectable. FIG. 11 illustrates the maxillary sinus template 56 in place on the bone model 30.

Referring to FIGS. 12-20, in another implementation, a maxillary framework 60 is shown, used for maxillary implants, completely supported by a patient's maxillary bone 12. Similar to the zygoma implant implementation, a segmented scan of a maxilla 12 is used to accurately map a maxilla framework 60 (FIGS. 15-20) that fits passively but snugly against the maxillary bone 12 surface. No screws are needed, but in other implementations screws may be used and added to the design for increased stabilization by including anchoring supports 46 and drill holes 48 as discussed above. The illustrated implementation shows a full arch design, but it is anticipated the design may also be configured as a half arch design if seating the full arch maxilla framework 60 is too difficult.

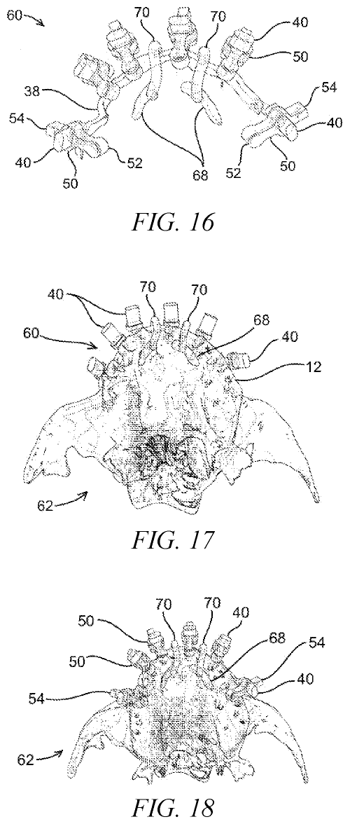

FIG. 12 illustrates a maxillary model scan 62, which is a scan model of a patient's initially scanned maxillary bone 12, after a surgeon has prepared the maxillary osteotomies 64 using a 3D printed model of the initially scanned maxillary bone 12 as discussed above. The osteotomies 64 are drilled where the surgeon proposes the patient's maxillary implants will be optimally placed, taking into consideration anatomic structures such as the maxillary sinus. Maxillary scan bodies 66 are placed in the maxillary model scan 62 which will then be used to create the maxilla framework 60 (FIGS. 14-20), position drill guide bushings 50 (FIGS. 16, 18, 19, 20), and thereby recreate the implant vectors that were pre-planned by the surgeon or technician on the 3D printed model of the patient's initially scanned maxillary bone 12.

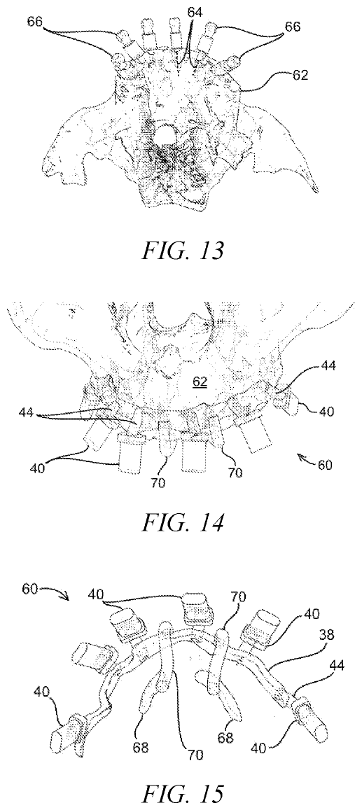

FIG. 13 illustrates a palatal view showing the maxillary scan bodies 66 in place in the maxillary osteotomies 64 as drilled by the surgeon on the 3D printed model of the maxillary initial scan 62. Alternatively, the procedure can be planned entirely using computer software that allows capture of a digitally placed implant. The software will then be used to place drill guide bushings (FIGS. 16, 18, 19, 20) to recreate the maxillary osteotomy 64 vectors prescribed by the surgeon. In this implementation, the maxillary scan bodies 66 are substantially similar to, and have the features of the zygoma scan bodies 20 (FIG. 2).

FIG. 14 illustrates a facial view of the maxillary framework 60 designed on the maxillary initial scan 62. As with other implementations, the maxillary framework 60 comprises a main support 38, from which extends at least one support armature 44. The support armatures 44 are each provided with a bushing support structure 40. The bushing support structures 40 are positioned in 3D design software relative to the implant vectors obtained from the prototype maxilla that was drilled by the treating surgeon.

FIG. 15 illustrates the maxillary framework 60 that will be used by the surgeon during the surgical implant placement procedure. The maxillary framework 60 can be 3D printed as a final device or may be cast using existing lost-wax casting techniques, or via any other modeling process. In addition to the support armatures 44 and bushing support structures 40, the main support 38 includes palatal pads 68 and palatal pad supports 70, which are used to secure the maxillary framework 60 on a patient's maxilla. The palatal pads 68 and palatal pad supports 70 are used in the maxillary framework 60 in lieu of the anchoring supports 46 which are used in the zygoma framework 36. Preferably the palatal pads 68 secure the maxillary framework 60 securely, requiring no fixation to the patient. Thus, there are no drill holes or other fixation features on the palatal pads 68. The maxillary framework 60 is also configured so as not to impinge on the naso-palatine nerve or the greater palatine vessel areas of a patient.

FIG. 16 illustrates the maxillary framework 60 with exchangeable drill guide bushings 50 in place on the bushing support structures 40. The drill guide bushings 50 can be designed with various drill diameters based on the surgical kit preference of the treating surgeon. The drill guide bushings 50 easily slide on and off the bushing support structures 40. A thread retainer portion 54 is provided on all drill guide bushings 50 to allow attachment of string or floss to allow easy retrieval in case they are dropped in the oral cavity, and a drill guide portion 52 is provided through which a dental bur extends.

FIG. 17 illustrates a palatal view of the maxillary framework 60 in place on a patient's maxilla, including palatal pads 68 and bushing support structures 40. It is assumed that the patient's gingival tissues (not shown) are reflected out of the way to seat the maxillary framework 60 in place.

FIG. 18 illustrates a palatal view of the maxillary framework 60 in place on a patient's maxilla, with bushing support structures 40 and interchangeable drill guide bushings 50 in place on the bushing support structures 40. A surgeon simply uses matching burs that fit the diameter of a particular drill guide bushing 50 to precisely drill the osteotomy from the prototype implant vector location.

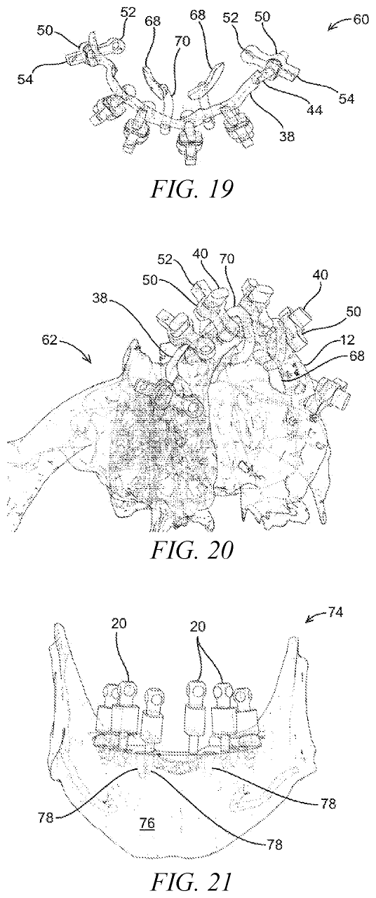

FIG. 19 illustrates the completed maxillary framework 60 inferior view, showing the main support 38, palatal pad supports 70 and palatal pads 68, and drill guide bushings 50 with the drill guide portions 52 and thread retainer portions. This shows an inferior view of the maxillary framework 60.

FIG. 20 illustrates a lateral oblique view of the maxillary framework 60 placed on the maxillary bone 12 showing the main support 38, palatal pads 68, palatal pad supports 70, and all drill guide bushings 50 in place.

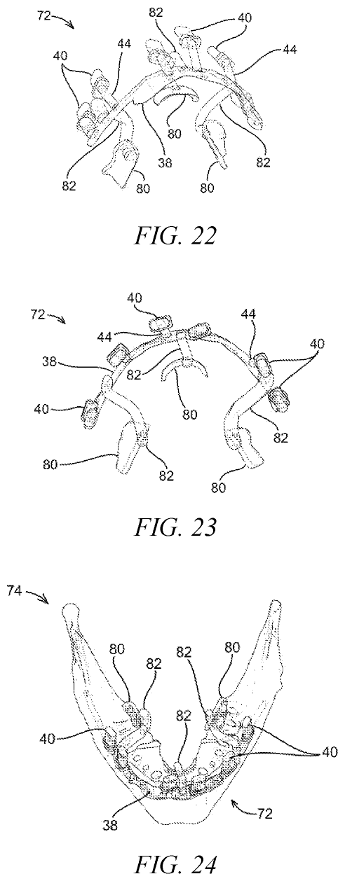

Referring to FIGS. 21-25, in another implementation, a mandibular framework 72 (i.e., mandibular bone supported) surgical guide is shown. The mandibular framework 72 is designed to be intimately adapted to the mandible bone 76 and is designed for implant placement in partially or fully edentulous cases. Screws (not shown) for support may be added by minor modification of the mandibular framework 72 (FIGS. 22-25) similar to the zygoma framework 36 in the above-described implementation, but the fit may be configured similar to the maxillary framework 60 mitigate the need for such screws simplifying its use during surgery. The design also allows maximum visualization of the surgical site and provides adequate opportunity for irrigation during drilling procedures. Other designs currently known in the art obscure the surgical field making irrigation and visualization difficult, whereas the mandibular framework 72 promotes adequate irrigation to prevent, for example, bone overheating.

Referring to FIG. 21, a mandibular scan body model 74 is a 3D printed jaw or computer representation showing planned implant vectors. The mandibular bone model 74 3D prototype is made by segmenting the mandibular bone 76 using the thresholding techniques or manual segmentation to separate bone from other tissues as described above. This segmentation is the output to suitable 3D printing software and hardware. Scan bodies 20 are shown in place in the mandibular osteotomies 78 which were drilled on a 3D stereolithographic print of the jaw structure. The scan bodies 20 are used to orient bushing support structures 40 that align with the scan bodies 20 and will allow attachment of drill guide bushings 50 that will align with the planned implant vectors. Avoidance of vital structures and thin bone can be readily accomplished using 3D printed models of adequate computer representations of the anatomic structures.

Referring to FIG. 22, a 3D representation of the designed mandibular framework 72 is shown, including lingual pads 80, lingual pad supports 82, and bushing support structures 40. In the illustrated implementation, the mandibular framework 72 includes three lingual pads 80 (and lingual pad supports 82) to provide an adequate intimate fit of the mandibular framework 72 directly on the mandibular bone 76. No screws are generally needed for this design, but drill holes 48 (FIG. 7) can be easily added to the lingual pads 80 if desired to secure the mandibular framework 72 to the mandibular bone 76.

Referring to FIG. 23, a top view of the finished mandibular framework 72 is shown. The mandibular framework 72 can be 3D printed in resin and used during the surgery or cast in metal using a lost wax technique, 3D printed in metal, or other methods. The illustration shows the three lingual pads 80, three lingual supports 82, and six bushing support structures 40.

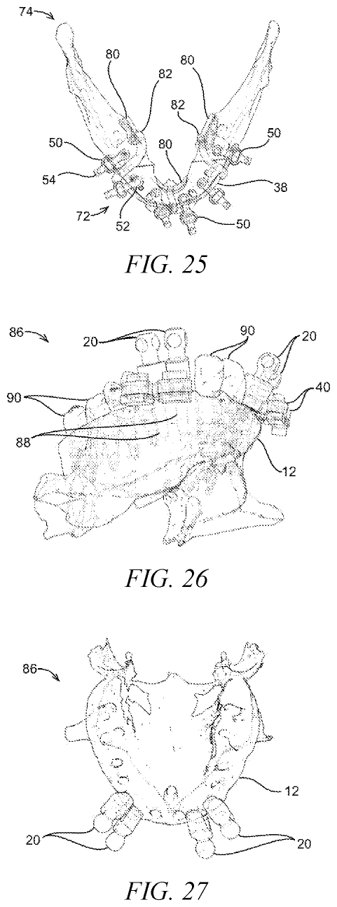

FIG. 24 illustrates the mandibular scan body model 74 with the mandibular framework 72 in place before installation of the drill guide bushings 50 (FIG. 25). The surgeon will then place various drill guide bushings to recreate the primarily designed implant vectors in an efficient manner.

FIG. 25 illustrates the mandibular scan body model 74 with the mandibular framework 72 mounted thereon. The illustration shows the mandibular framework 72 in place on a jaw that was previously alveoloplastied to provide prosthetic space. The main support 38 and lingual pads 80 are used for stable mandibular bone 76 support. The drill guide bushings 50 are placed on the bushing support structures 40. The bushing support structures 40 are attached to mandibular framework 72 with support armatures 40. The removeable drill guide bushings 50 can be designed with different diameters to accept any drill or osteotome.

Referring to FIGS. 26-29, in another implementation a tooth supported framework 84 is shown. The tooth supported framework 84 allows removable drill guide bushings 50 to be used to replicate implant placement as prepared on a 3D printed model (not shown). The drilled 3D model is then scanned to create a tooth supported framework scan 86 with scan bodies 20 that are placed directly within the prepared tooth supported framework osteotomies 88. The 3D model with the scan bodies 20 is scanned in a dental scanner and 3D representations of the scan bodies 20 are aligned accurately to capture the pre-planned implant vectors. This design allows optimal surgical field visualization and greater ability for irrigation that other designs do not allow due to the bulk and position of conventional drill sleeves. The drill guide bushings 50 (FIG. 29) can be designed of various angles and length. The drill guide bushings 50 is designed to accept any implant diameter that the surgeon desires.

FIG. 26 illustrates a segmented, tooth supported framework scan 86 having maxillary bone 12 and teeth 90 structures. This data is used to print a 3D model using stereolithography and the surgeon can then drill optimal implant positions. Scan bodies 20 are placed with the tooth supported framework osteotomies 88 and implant vectors are obtained by scanning the model in a dental scanner that has the scan bodies in place. 3D scan bodies 20 are then aligned which allow support structures 40 to be placed, which will match one or more drill guide bushings 50. This process takes the prototype data and allows it to be transferred digitally to then produce a tooth supported framework 84 surgical guide and used to place the real dental implants during actual surgical procedure.

FIG. 27 illustrates a 3D model with teeth removed and showing scan bodies 20 in place. This is scanned in a dental scanner and 3D representation of the scan bodies 20 are aligned to create the tooth supported framework scan 86. This will allow for the tooth supported framework 84, and related support structures discussed below to be placed, to reproduce planned implant vectors.

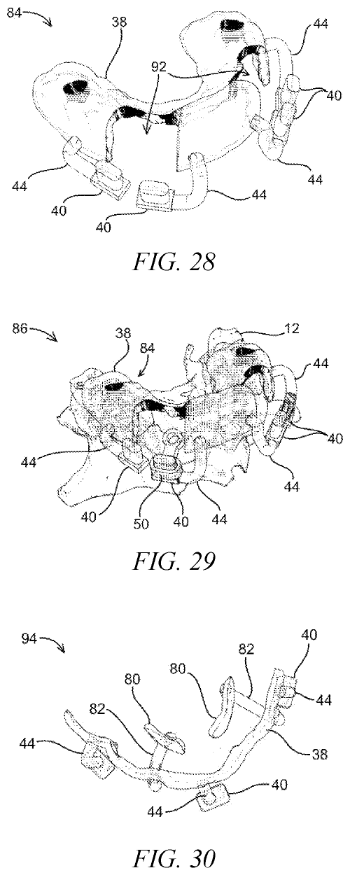

FIG. 28 illustrates a top view of completed printed tooth supported framework 84 showing drill guide bushing 50 (FIG. 29) bushing support structures 40 and support armatures 44 attaching the bushing support structures 40 to the main support 38 that is tooth supported. Cutouts 92 are also added to ensure the tooth supported framework 84 is seated adequately. This design allows optimal view of the surgical site and allows irrigation at the penetration site of the drill into the bone thus limiting bone heating which increases implant survival.

FIG. 29 illustrates a finished tooth supported framework 84, showing the main support 38, which in this instance is an impression-based tooth support structure, mounted on the maxillary bone 12 of the tooth supported framework scan 86. Several bushing support structures 40 are provided mounted on the main support 38 using support armatures 44. A single drill guide bushing 50 can be easily removed and replaced with various other drill guides bushings 50 of various diameters to be used during the implant placement sequence. This design allows optimal visualization of the surgical site and allows easy irrigation to cool the implant burs and prevent bone overheat which could lead to increased implant failure. This device is usually 3D printed using resin and stereolithography techniques or other similar production techniques.

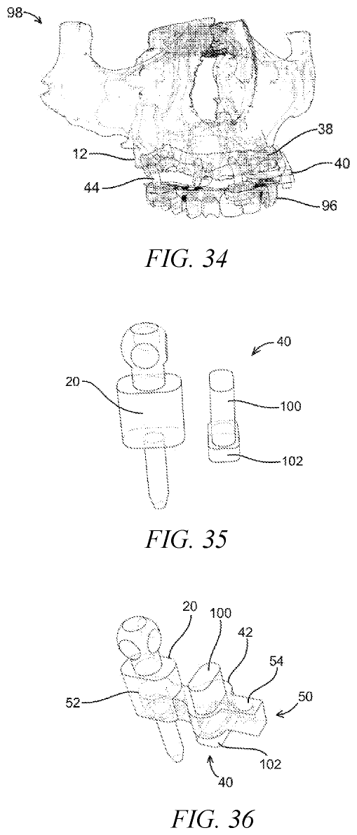

Referring to FIGS. 30-33, in another implementation, a bite prosthetic jig 94 is shown and described. The bite prosthetic jig 94 device is designed to provide the bite prosthetic 96 (FIGS. 32-33). when used during a major implant reconstruction. In this example all upper teeth were removed from the patient's maxilla 12, implants were placed using guided procedure described below, and a framework and bite prosthetic jig 94 replacing teeth and gingiva was designed using software. The bite prosthetic jig 94 is fitted to the patient jawbone structure, such as the maxilla 12 (FIGS. 33, 34) in one piece. A second piece, the bite prosthetic 96 can then be placed as a separate piece. The bite prosthetic jig 94 is only used in this case to support the prosthetic device 96 and nothing else. Once the bite prosthetic jig 94 and prosthetic device 96 are in place the surgeon can capture this bite by luting it to the dental implants. Screws are removed from temporary cylinders placed on the dental implants and the top prosthetic device is lifted from the bite prosthetic jig 94. The bite prosthetic jig 94 is then removed as a separate piece. The bite prosthetic jig 94 allows capturing the 3D points of interest for prosthetic reconstruction including the incisal edge point, tooth distribution, and bite plane for instance Camper's plane as is used in this example. These 3D positions are referenced directly to the bone to form a stable base.

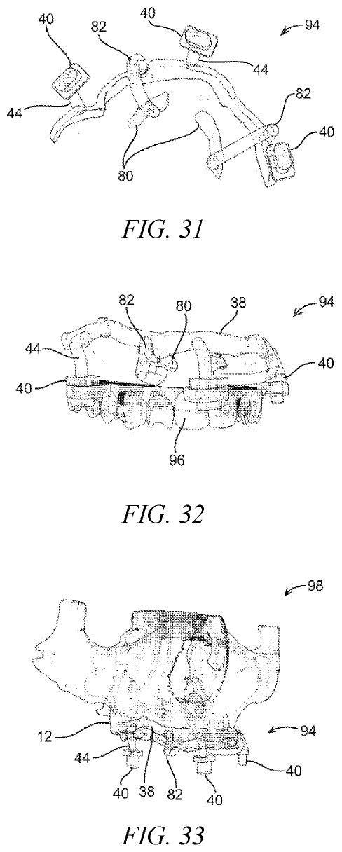

FIG. 30 illustrates an inferior view of a bite prosthetic jig 94 that includes lingual pad 80 and lingual pad supports 82 for fixing the bite prosthetic jig 94 on the patient. Also included are three bushing support structures 40 mounted on support armatures 44 connecting them to a main support 38. FIG. 31 illustrates a superior view of the bite prosthetic jig 94.

FIG. 32 illustrates the bite prosthetic jig 94 and bite prosthetic 96 assembled. The bone-supported bite prosthetic jig 94 has three bushing support structures 40, and the bite prosthetic 96 can be made of acrylic and 3D printed is shown mated to the bite prosthetic jig 94. The bite prosthetic jig 94 can be 3D printed and made of various materials such as resin or metal.

FIG. 33 illustrates a bite prosthetic jig scan 98 of a patient's maxilla 12 with the bite prosthetic jig 94 affixed thereto.

FIG. 34 illustrates the bite prosthetic jig scan 98 showing the bite prosthetic jig 94 attached to the patient's maxilla 12, with the bite prosthetic 96 mounted thereto. Once the bite prosthetic 96 is in place, the bite prosthetic 96 is luted to cylinders (not shown) placed on previously inserted implants thus relating the bite prosthetic 96 to the dental implants. The cylinders are unscrewed from the implants and the bite prosthetic jig 94 and implant cylinder are removed in one luted piece. The bite prosthetic jig 94 is then removed as a separate piece.

FIG. 35 illustrates a scan body 20 and bushing support structure 40 in alignment. Once the scan body 20 is aligned with the prototype surgery (i.e., once the osteotomies are drilled in the initial scan model), the bushing support structure 40 can be placed in correct 3D position. In the illustrated implementation, the bushing support structure 40 includes a male support structure 100 and a support base 102.

FIG. 36 illustrates a drill guide bushing 50 in place on a bushing support structure 40. In addition to the drill guide portion 52 and the thread retainer portion 54, the drill guide bushing has a support structure slot 42 configured to seat over the male support structure 102 and rest against the support base 102. Thus, with the drill guide bushing aligned with both the scan body 20 and the bushing support structure 40, the drill guide portion 52 is aligned with each osteotomy planed by the surgeon on the initial scan model.

The foregoing descriptions of embodiments of the present invention have been presented only for purposes of illustration and description. They are not intended to be exhaustive or to limit the present invention to the forms disclosed. Accordingly, many modifications and variations will be apparent to practitioners skilled in the art. Additionally, the above disclosure is not intended to limit the present invention. The scope of the present invention is defined by the appended claims.

What is claimed is:

1. An osteotomy based scan body apparatus for an accurately planned dental implant vector on a bone implant site in patient bone, the apparatus comprising:
a main support member configured to rest adjacent the patient bone; a bushing support structure extending from the main support member, resiliently connected to the main support member, and extending away from the patient bone; a drill guide bushing having a support structure slot and a drill guide portion; wherein the support structure slot is configured to engage the bushing support structure non-rotationally; and wherein the drill guide portion comprises a loop member comprising a loop encircling the implant vector, the loop configured for receiving a dental bur.

2. The apparatus of claim 1 further comprising at least one anchoring support affixed to the main support member, the anchoring support configured to engage the patient bone, thereby anchoring the main support member in place relative to the patient bone.

3. The apparatus of claim 2 wherein the at least one anchoring support comprises at least one drill hole for fixing the anchoring support to the patient bone.

4. The apparatus of claim 1 wherein the bushing support structure is affixed to the main support member using a support armature configured to space the bushing support structure away from the main support member.

5. The apparatus of claim 1 wherein the bushing support structure comprises a male support structure for engaging the support structure slot, and a support base.

6. The apparatus of claim 5 wherein the bushing support structure is configured such that the support structure slot cannot slide past the support base.

7. The apparatus of claim 1 wherein the drill guide bushing further comprises a thread retainer portion.

8. The apparatus of claim 7 wherein the thread retainer portion is disposed opposite the drill guide portion of the drill guide bushing.

9. The apparatus of claim 7 wherein the thread retainer portion is disposed laterally relative to the drill guide portion of the drill guide bushing.

10. The apparatus of claim 1 further comprising a maxillary sinus template having template windows for locating maxillary sinus windows on the patient bone.

11. The apparatus of claim 1 wherein the main support member is configured to abut the outside arch of the patient's maxillary bone.

12. The apparatus of claim 11 further comprising at least one palatal pad engaging the patient's maxillary bone opposite the main support member, thereby anchoring the main support member.

13. The apparatus of claim 12 further comprising at least one palatal pad support connecting the at least one palatal pad to the main support member.

14. The apparatus of claim 1 wherein the main support member is configured to abut the outside arch of the patient's mandibular bone.

15. The apparatus of claim 14 further comprising at least one lingual pad engaging the patient's mandibular bone opposite the main support member, thereby anchoring the main support member.

16. The apparatus of claim 15 further comprising at least one lingual pad support connecting the at least one lingual pad to the main support member.

17. The apparatus of claim 1 wherein the main support member is a tooth supported framework configured to seat over a patient's existing teeth.

18. The apparatus of claim 17 wherein the tooth supported framework comprises at least one cutout at the location of the dental implant vector.

19. The apparatus of claim 1 wherein the main support member is a bite prosthetic jig configured to engage a patient's maxillary bone after the removal of all teeth.

20. The apparatus of claim 19 further comprising a bite prosthetic.
